(19) United States
(12) Patent Application Publication
(10) Pub. No.: US 2001/0002254 A1
(43) Pub. Date: May 31, 2001

(54) Compositions and methods for coloring hair

(75) Inventors: Dalal Ibrahim Duffer, North Brunswick, NJ (US); Teresita Vergaria Imperial, Staten Island, NY (US)

Correspondence Address: Julie Blackburn, Esq., Revlon Consumer Products Corporation, Law Dept., 625 Madison Avenue, New York, NY, 10022, US

(73) Assignee: Revlon Consumer Products Corporation, New York, NY (US)

(21) Appl. No.: 09/768,485

(22) Filed: Jan. 25, 2001

Related U.S. Application Data
(63) Continuation of application No. 09/213,794, filed on Dec. 17, 1998, now abandoned.
(60) Provisional application No. 60/099,473, filed on Sep. 8, 1998.

(51) Int. Cl. A61K 7/06; A61K 7/075; A61K 7/11; A01N 39/00; A01N 31/14; A61K 31/08; A61K 33/40; A61K 7/08; A61K 7/13

(52) U.S. Cl. 424/70.1; 424/70.6; 424/70.12; 424/70.16; 424/70.31; 424/613; 424/616; 514/723; 514/724; 514/549; 8/407; 8/429

(57) ABSTRACT

A developer composition having a pH of 2.5 to 6.9, for use in oxidative dyeing of hair comprising 0.01-20% of a primary thickening agent which is an aqueous solution of acrylic polymer comprised of A and B monomers wherein the A monomer is selected from the group consisting of acrylic acid, methacrylic acid, and mixtures thereof; and the B monomer is selected from the group consisting of a C₁₋₂₂ alkyl methacrylate, and a C₁₋₂₂ alkyl acrylate, and mixtures thereof; 0.01-20% of a nonionic surfactant, 0.01-25% hydrogen peroxide, and 65-99.9% water; a hair dye mixture for coloring hair; a two component kit for coloring hair; and a method for coloring hair.

### RELATED APPLICATIONS

This application is a continuation-in-part of provisional patent application Ser. No. 60/099,473, filed September 9, 1998.

### TECHNICAL FIELD

The invention is in the field of compositions and methods for coloring hair.

### BACKGROUND OF THE INVENTION

A large majority of women over the age of forty color their hair. Oxidative hair dyes provide permanent coloration to hair, lasting from four to six weeks. They are usually sold in the form of a two component kit. In one container is a lotion that contains the hair dye molecules and other ingredients in an aqueous system. In the other container is a developer solution which contains an oxidizing agent, usually hydrogen peroxide. The two components are mixed immediately prior to use. The dye precursors in the lotion will penetrate the hair and become oxidized by the oxidizing agent to produce color in the hair. The mixture is left on the hair for an appropriate period of time, generally 5 to 60 minutes, then rinsed off with water. Obviously, the hair dye mixture which results when the lotion and developer are mixed must have the appropriate Theological properties. In other words, the mixture must be thick enough so that it can be applied to the hair without excessive dripping. On the other hand, the mixture must not be so thick that it cannot easily be removed by rinsing with water after completion of the dyeing process. Many commercial dye lotion and developer combinations contain nonoxynol as a thickening agent. Often the nonoxynol is found in the developer solution. When the dye lotion and developer are mixed the nonoxynol thickens the mixture to the desired rheology. Unfortunately, however, while nonoxynol is an excellent thickener, it is believed to have certain undesireable properties, most notably, at very high concentrations it is a spermicide. Thus, manufacturers of hair dye products are attempting to make products which reduce or eliminate the use of nonoxynol as a thickening agent yet have the proper rheological properties.

It is an object of the invention to make a developer composition capable of thickening a hair dye lotion mixed with the developer composition to the desired rheology.

It is an object of the invention to make a developer composition free of nonoxynol.

It is a further object of the invention to make a dye/developer mixture free of alkyl sulfate surfactants, alkyl ether sulfate surfactants, amphoteric surfactants, zwitterionic surfactants.

It is an object of the invention to provide a method for oxidative dyeing of hair using a hair dye/developer mixture that exhibits desireable rheology and mildness.

### SUMMARY OF THE INVENTION

The invention comprises a developer composition for use in oxidative dyeing of hair having a pH of 2.5 to 6.9, comprising, by weight of the total composition:

0.01-20% of a primary thickening agent which is an aqueous solution of an acrylic polymer comprised of A and B monomers wherein the A monomer is selected from the group consisting of acrylic acid, methacrylic acid, and mixtures thereof; and the B monomer is selected from the group consisting of a C₁₋₂₂ alkyl methacrylate, and a C₁₋₂₂ alkyl acrylate and mixtures thereof,

0.01-20% of a nonionic surfactant,

0.01-25% hydrogen peroxide,

65-99.9% water.

The invention further comprises a mixture for oxidation dyeing of hair comprising, by weight of the total composition:

(a) about 0.0001-20% (combined weight) of at least one primary intermediate and at least one coupler for the formation of oxidation dyes,

(b) about 0.5-30% of a nonionic surfactant,

(c) about 0.01-20% of a primary thickening agent which is an acrylic copolymer comprised of monomers A and B wherein the A monomer is selected from the group consisting of acrylic acid, methacrylic acid, and mixtures thereof; and the B monomer is selected from the group consisting of a C₁₋₂₂ alkyl methacrylate, and a C₁₋₂₂ alkyl acrylate and mixtures thereof; and

(d) about 40-99% water.

The invention further comprises a two component kit for oxidative dyeing of hair comprising:

(a) an aqueous lotion having a pH of 7 to 11, comprising, by weight of the total composition:

(i) about 0.0001-20% (combined weight) of at least one primary intermediate and at least one coupler for the formation of oxidation dyes,

(ii) about 0.5-20% of a nonionic surfactant, and

(iii) about 30-90% water.

(b) a developer composition having a pH of 2.5 to 6.9, comprising, by weight of the total composition:

0.01-20% of a primary thickening agent which is an aqueous solution of an acrylic polymer comprised of A and B monomers wherein the A monomer is selected from the group consisting of acrylic acid, methacrylic acid, and mixtures thereof, and the B monomer is selected from the group consisting of a C₁₋₂₂ alkyl methacrylate, and a C₁₋₂₂ alkyl acrylate and mixtures thereof;

0.01-20% of a nonionic surfactant,

0.01-25% hydrogen peroxide,

5 65-99.9% water.

The invention also comprises a method for oxidative dyeing of hair comprising the steps of:

(a) applying to the hair a composition obtained by mixing Composition A and Composition B, wherein Composition A is an aqueous lotion comprising, by weight of the total composition:

(i) about 0.0001-20% (combined weight) of at least one primary intermediate and at least one coupler for the formation of oxidation dyes,

(ii) about 0.5-20% of a nonionic surfactant, and

(iii) about 10-65% water.

and Composition B is a developer composition comprising, by weight of the total composition:

(i) about 0.5-45% hydrogen peroxide,

(ii) 0.01-20% of a primary thickening agent comprised of an aqueous solution of acrylic copolymer comprised of monomers A and B wherein the A monomer is selected from the group consisting of acrylic acid, methacrylic acid, and mixtures thereof; and the B monomer is selected from the group consisting of a C₁₋₂₂ alkyl methacrylate, and a C₁₋₂₂ alkyl acrylate and mixtures thereof;

(iii) 0.01-20% of a nonionic surfactant,

(iv) 65-99.9% water.

b) leaving said composition on the hair for 2 to 60 minutes,

c) rinsing the hair with water.

### DETAILED DESCRIPTION

All percentages mentioned herein are percentages by weight unless otherwise indicated.

The Developer Composition

The invention first comprises a developer composition for use in oxidative coloration of hair. The composition comprises a variety of ingredients as set forth herein and has a pH of 2.5 to 6.9, preferably 3 to 6, more preferably 3 to 5. Preferably, the developer composition has a viscosity of about 10 to 200 centipoise, more preferably 10-100 centipoise at 25°C.

### THE PRIMARY THICKENING AGENT

The developer composition comprises 0.01-20%, preferably 0.05-15%, more preferably 0.1-10% by weight of a primary thickening agent which is an aqueous solution of an acrylic polymer comprised of monomers A and B wherein A is selected from the group consisting of acrylic acid, methacrylic acid, and mixtures thereof; and B is selected from the group consisting of a C₁₋₂₂ alkyl acrylate, a C₁₋₂₂ alky methacrylate, and mixtures thereof. Preferably, the A monomer comprises one or more of acrylic acid or methacrylic acid, and the B monomer comprises is selected from the group consisting of a C₁₋₁₀, most preferably C₁₋₄ alkyl acrylate, a C₁₋₁₀, most preferably C₁₋₄ alkyl methacrylate, and mixtures thereof. Most preferably the B monomer is one or more of methyl or ethyl acrylate or methacrylate. Most preferably, the acrylic copolymer is supplied in an aqueous solution having a solids content ranging from about 10-60%, preferably 20-50%, more preferably 25-45% by weight of the polymer, with the remainder water. The composition of the acrylic copolymer may contain from about 0. 1-99 parts of the A monomer, and about 0.1-99 parts of the B monomer. Preferably, the acrylic copolymer contains enough of the A monomer to enable ionization in a basic solution, thereby causing the ionized carboxylic acid groups in the polymer to repel each other, and thereby “swallow” water. Particularly preferred acrylic copolymer solutions suitable for use in the developer composition include those sold by Seppic, Inc., under the tradename Capigel, in particular, Capigel 98, which is a white liquid having a pH of 2 to 4, a solids content of about 29-31, a density of 1.04 to 1.08, and a viscosity of 700-1000 millipascal seconds at 25°C.

### NONIONIC SURFACTANT

The developer composition further comprises 0.01-20%, preferably 0.05-15%,more preferably 0.1-10% by weight of a nonionic surfactant. Nonionic surfactants are generally compounds produced by the condensation of alkylene oxide groups with a hydrophobic compound. Examples of nonionic surfactants are:

(a) Long chain dialkyl sulfoxides containing one short chain alkyl or hydroxy alkyl radical of from about 1 to 3 carbon atoms and one long hydrophobic chain which may be an alkyl, alkenyl, hydroxyalkyl, or ketoalkyl radical containing from about 8 to 20 carbon atoms, from 0 to 10 ethylene oxide moieties, and 0 or 1 glyceryl moiety.

(b) Polysorbates, such as sucrose esters of fatty acids. Examples of such materials include sucrose cocoate, sucrose behenate, and so on.

(c) Polyethylene oxide condensates of alkyl phenols, for example the condensation products of alkyl phenols having an alkyl group of 6 to 20 carbon atoms with ethylene oxide being present in amounts of about 10 to 60 moles of ethylene oxide per mole of alkyl phenol.

(d) Condensation products of ethylene oxide with the reaction product of propylene oxide and ethylene diamine.

(e) Condensation products of aliphatic alcohols having 8 to 18 carbon atoms with ethylene oxide, for example a coconut alcohol/ethylene oxide condensate having 10 to 30 moles of ethylene oxide per mole of coconut alcohol, the coconut alcohol fraction having 10 to 14 carbon atoms.

(f) Long chain tertiary amine oxides such as those corresponding to the general formula:

R₁R₂R₃NO

wherein R₁ contains an alkyl, alkenyl or monohydroxyalkyl radical ranging from about 8 to 18 carbon atoms in length, from 0 to about 10 ethylene oxide moieties, and from 0 to about 1 glyceryl moiety and R₂ and R₃ are each alkyl or monohydroxyalkyl groups containing from about 1 to about 3 carbon atoms.

(g) Long chain tertiary phosphine oxides corresponding to the general formula:

RR₁R₂PO

wherein R contains an alkyl, alkenyl, or monohydroxyalkyl radical having 8 to 18 carbon atoms, from 0-10 ethylene oxide moieties and 0 or 1 glyceryl moiety, and R₂ and R₃ are each alkyl or monohydroxyalkyl group containing from about 1 to 3 carbon atoms.

(h) Polyethylene glycol (PEG) glyceryl fatty esters, having the formula

RC(O)OCH₂CH(OH)CH₂(OCH₂CH₂)ₙOH

wherein n is 5-200 and RC(O)— is a hydrocarbylcarbonyl group wherein R is preferably an aliphatic radical having 7 to 19 carbon atoms.

(i) C₁₀₋₁₈ alkyl(C₁₋₆)polyhydroxy fatty acid amides such as C₁₂₋₁₈methylglucamides, N-alkoxy polyhydroxy fatty acid amides, N-propyl through N-hexyl C₁₂₋₁₈glucamides and so on.

(j) Alkyl polysaccharides having a hydrophobic group of 6 to 30, preferably 10, carbon atoms and a polysaccharide group such as glucose, galactose, etc. Suitable alkyl polysaccharides are octyl, nonydecyl, undecyldodecyl, tridecyl, tetradecyl, pentadecyl, hexadecyl, heptadecyl, and octadecyl, di-, tri-, tetra-, penta-, and hexaglucosides, galactosides, lactosides, glucoses, fructosides, fructoses, and so on.

Preferred nonionic surfactants are alkyl polysaccharides, or alkyl glycosides, disclosed in U.S. Pat. Nos.5,716,418 and 5,756,079, both of which are hereby incorporated by reference. These alkylglycosides have the general formula:

R₁—O—(R₂O)ₜ—H

wherein R₁ is a linear or branched alkyl or alkenyl radical having 12 to 30 carbon atoms, R₂ is a C₂₋₄ alkylene, (G) is an anhydroglucose unit, t is a number between 0 and 10, preferably 0 to 4, and n is a number from about 1 to 15. These alkyl polyglycosides are sold by Henkel Corporation under the tradenamnes APG 300, APG 350, APG 500, APG 550, APG 625, or the tradename Planteren, e.g. Planteren 300, 600, 1200, 2000, and so on. Particularly preferred is where R₁ is a linear radical having 16 carbon atoms, e.g. lauryl; t is 0; G is anhydroglucose; and n is 1 to 5. Such alkyl glycosides are sold by Henkel Corporation under the tradename Planteren 1200, which is a fatty polyglycoside prepared by reacting corn starch glucose with a C12-16 natural fatty alcohol, having the CTFA name lauryl polyglucose.

The remainder of the developer composition is water, in particular, about 40-99% by weight. Preferably, the developer composition is free of nonoxynol, as well as other anionic surfactants such as alkyl sulfates or alkyl ether sulfates, amphoteric and/or zwitterionic surfactants.

### OTHER INGREDIENTS

The developer composition may contain other ingredients such as secondary thickening agents, preservatives, chelating agents, conditioning agents, pH adjusters, and so on.

In the preferred embodiment of the invention, the developer composition contains 0.01-10%, preferably 0.05-8%, more preferably 0.1-5% of a secondary thickening agent comprised of polymer of A, B, and C monomers wherein A and B are as defined above, and C has the general formula:

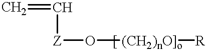

wherein Z is —(CH₂)ₘ— or

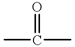

wherein m is 1-10, n is 2-3, o is 2-200, and R is a C10-30 straight or branched chain alkyl. Examples of the secondary thickening agent above, are copolymers where A and B are defined as above, and C is CO, and wherein n, o, and R are as above defined. Examples of such secondary thickening agents include acrylates/steareth-20 methacrylate copolymer, which is sold by Rohm & Haas under the tradename Acrysol ICS-.

Preferably, in the copolymer used for the secondary thickening agent in the preferred embodiment of the invention, A and B are as above defined; and in the C monomer Z is (CH₂)ₘ, m is 1-2, n is 2, and o is 2-100, and R is a C₁₂₋₂₂ straight or branched chain alkyl. More preferably in the C monomer m is 1, n is 2, o is 10, and R is C₁₈ or stearyl, and the compound is steareth-10 allyl ether/acrylate copolymer, which may be purchased from Allied Colloids under the tradename Salcare SC90.

The developer composition may also contain nonionic conditioning agents which provide a subtle conditioning effect to the hair. Suitable conditioning agents include silicones, and suggested ranges are 0.001-10%, preferably 0.005-8%, more preferably 0.01-5% by weight. Suitable silicone hair conditioning agents include volatile or nonvolatile nonionic silicone fluids, silicone resins, and silicone semi-solids or solids.

Volatile silicones are linear or cyclic silicones having a measureable vapor pressure, which is defined as a vapor pressure of at least 2 mm. of mercury at 20°C. Examples of volatile silicones are cyclic silicones having the general formula:

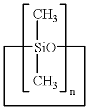

where n 3-7.

Also, linear volatile silicones that may be used in the compositions of the invention have the general formula:

(CH₃)₃Si—O—[Si(CH₃)₂—O]ₙ—Si(CH₃)₃

where n=0-7, preferably 0-5.

The silicone hair conditioning agent may comprise water insoluble nonvolatile silicone fluids including polyalkyl siloxanes, polyaryl siloxanes, polyalkylaryl siloxanes, polyether siloxane copolymers, amine-functional silicones, and mixtures thereof Such silicones have the following general formula:

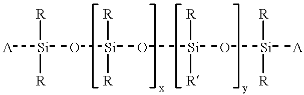

wherein R and R′ are each independently alkyl, aryl, or an alkyl substituted with one or more amino groups, and x and y are each independently 0-100,000, with the proviso that x+y equals at least one and A is siloxy endcap unit. Preferred is where A is methyl, R is methyl, and R′ is an alkyl substituted with at least two amino groups, most preferably an amine-functional silicone having the formula:

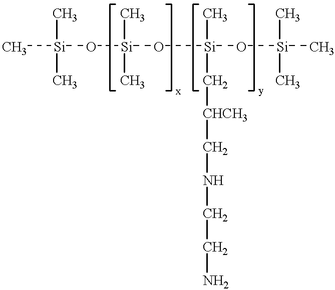

which is known by the CTFA name trimethylsilylamodimethicone.

The silicone hair conditioning agent may also be a silicone polymer having the following general formula: x y

wherein R, R′ and R″ are each independently a C₁₋₁₀ straight or branched chain alkyl or phenyl, and x and y are such that the ratio of (RR′R″)₃SiO_{½}units to SiO₂ units is 0.5 to 1 to 1.5 to 1.

Preferably R, R′ and R″ are a C₁₋₆ alkyl, and more preferably are methyl and x and y are such that the ratio of (CH₃)₃SiO_{½}units to SiO₂ units is 0.75 to 1. Most preferred is this trimethylsiloxy silicate containing 2.4 to 2.9 weight percent hydroxyl groups which is formed by the reaction of the sodium salt of silicic acid, chlorotrimethylsilane, and isopropyl alcohol. The manufacture of trimethylsiloxy silicate is set forth in U.S. Pat. Nos. 2,676,182; 3,541,205; and 3,836,437, all of which are hereby incorporated by reference. Trimethylsiloxy silicate as described is available from Dow Corning Corporation under the tradename 2-0749 and 2-0747 (also referred to as Dow Corning 749 Fluid) each of which is a blend of about 40-60% volatile silicone and 40-60% trimethylsiloxy silicate. Dow Corning 2-0749, in particular, is a fluid containing about 50% trimethylsiloxy silicate and about 50% cyclomethicone. The fluid has a viscosity of 200-700 centipoise at 25°C., a specific gravity of 1.00 to 1.10 at 25°C., and a refractive index of 1.40-1.41.

Preferably the developer composition contains 0.001-10% trimethylsilylamodimethicone which is purchased in an aqueous emulsion form as a mixture comprising, by weight of the total emulsion mixture, 10-30% trimethylsilylamodimethicone, 1-5% C11-15 pareth-7, 1-5% laureth-9, 1-5% trideceth-12, 1-5% glycerin and 60-80% water, which is sold by GE Silicones under the tradename GE SME253.

The composition may also contain 0.0001-7%, preferably 0.001-5%, more preferably 0.005-3% preservatives. Suitable preservatives include methyl, ethyl, and propyl paraben, DMDM hydantoin, DEDM hydantoin, benzyl alcohol, and the like.

The composition may also contain 0.0001-5%, preferably 0.0005-3%, more preferably 0.001-2% of one or more chelating agents which are capable of complexing with and inactivating metallic ions in order to prevent their adverse effects on the stability or effects of the composition. In particular, the chelating agent in the developer will chelate the metal ions found in the water and prevent these ions from interfering with the reaction between the dye molecules and the oxidizing agent. Suitable chelating agents include EDTA and calcium, sodium, or potassium derivatives thereof, HEDTA, sodium citrate, TEA-EDTA, and so on.

It may also be desireable to add small amounts of acids or bases to adjust the pH of the developer composition to the desired 7 to 11. Suitable acids include hydrochloric acid, phosphoric acid, citric acid, and the like. Suitable bases include sodium hydroxide, potassium hydroxide, and the like.

Hair Dye Mixture

The invention further comprises a mixture for oxidation dyeing of hair comprising, by weight of the total composition:

0.0001-20% (combined weight) of at least one primary intermediate and at least on coupler for the formation of oxidation dyes,

0.5-30% of a nonionic surfactant,

0.01-20% of a primary thickening agent which is an aqueous solution of acrylic polymer comprised of monomers A and B, wherein the A monomer is selected from the group consisting of acrylic acid, methacrylic acid, and mixtures thereof; and the B monomer is selected from the group consisting of a C₁₋₂₂ alkyl methacrylate, C₁₋₂₂ alkyl acrylate, and mixtures thereof; and

40-99% water.

The hair dye mixture is formed by combining a hair dye lotion which contains the dye molecules in aqueous solution, with the developer in a ratio ranging from about 1 to 2 parts lotion to 1 to 2 parts developer. When the acidic pH developer is mixed with the basic pH lotion, the mixture is neutralized, thereby causing ionization of the aqueous acrylic polymer, which causes the substituent chains in the polymer to repel each other and provides the effect of “swallowing” water. This, in turn, causes gelling of the mixture to the appropriate rheology, which is, preferably, a viscosity ranging from about 2,000 to 5,000 centipoise, more preferably 2,800 to 4,800 centipoise, most preferably about 2800 to 3500 centipoise at 25°C. The nonionic surfactant and primary thickening agent found in the mixture are as mentioned above with respect to the developer composition.

### PRIMARY INTERMEDIATES AND COUPLERS

The hair dye mixture comprises from about 0.0001-20%, preferably 0.001-15%, more preferably 0.005-12% (combined weight) of primary intermediates and couplers. These ingredients are well known for use in hair color, and include ortho or para substituted aminophenols or phenylenediamines, such as para-phenylenediamines of the formula:

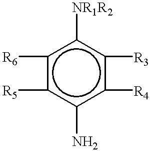

wherein R₁ and R₂ are each independently hydrogen, C₁₋₆ alkyl, or C₁₋₆ alkyl substituted with one or more hydroxy, methoxy, methylsulphonylamino, aminocarbonyl, furfuryl, unsubstituted phenyl, or amino substituted phenyl groups; R₃, R₄, R₅, and R₆ are each independently hydrogen, C₁₋₆ alkyl, C₁₋₆ alkoxy, halogen, or C₁₋₆ alkyl substituted with one or more hydroxy or ammo groups.

Examples of suitable primary intermediates include para-phenylenediamine, 2-methyl-1,4-diaminobenzene, 2,6-dimethyl-1,4-diaminobenzene, 2,5-dimethyl-1,4-diaminobenzene, 2,3-dimethyl-1,4-diaminobenzene, ortho-aminophenol, 2-chloro-1,4-diaminobenzene, 2-methoxy-1,4-diaminobenzene, 1-phenylamino-4-aminobenzene, 1-dimethylamino-4-aminobenzene, 1 -diethylamino-4-aminobenzene, 1-bis(beta-hydroxyethyl)amino-4-aminobenzene, 1-methoxyethylamino-4-aminobenzene, 2-hydroxymethyl-1,4-diaminobenzene, 2-hydroxyethyl-1,4-diaminobenzene, 2-isopropyl-1,4-diaminobenzene, 1-hydroxypropylamino-4-aminobenzene, 2,6-dimethyl-3 -methoxy- 1,4-diaminobenzene, 1-amino-4-hydroxybenzene, and derivatives thereof, and acid or basic salts thereof.

Preferred primary intermediates are p-phenylenediamine, p-aminophenol, o-aminophenol, N,N-bis(2-hydroxyethyl)-p-phenylenediamine, 2,5-diaminotoluene, their salts and mixtures thereof.

Suitable couplers include, for example, those having the general formula:

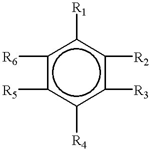

wherein R₁ is unsubstituted hydroxy or amino, or hydroxy or amino substituted with one or more C₁₋₆hydroxyalkyl groups, R₃ and R₅ are each independently hydrogen, hydroxy, amino, or amino substituted with C₁₋₆ alkyl, C₁₋₆ alkoxy, or C₁₋₆ hydroxyalkyl group; and R₂, R₄, and R₆ are each independently hydrogen, C₁₋₆ alkoxy, C₁₋₆ hydroxyalkyl, or C₁₋₆ alkyl, or R₃ and R₄together may form a methylenedioxy or ethylenedioxy group. Examples of such compounds include meta-derivatives such as phenols, catechol, meta-aminophenols, meta-phenylenediamines, and the like, which may be unsubstituted, or substituted on the amino group or benzene ring with alkyl, hydroxyalkyl, alkylamino groups, and the like. Suitable couplers include 3,4-methylenedioxyphenol, 3,4-methylenedioxy-1-[(beta-hydroxyethyl)amino]benzene, 1 -methoxy-2-amino-4-[(beta-hydroxyethyl)amino]benzene, 1 -hydroxy-3-(dimethylamino)benzene, 6-methyl-1-hydroxy-3[(beta-hydroxyethyl)amino]benzene, 2,4-dichloro-1-hydroxy-3-aminobenzene, 1 -hydroxy-3 -(diethylamino)benzene, 1-hydroxy-2-methyl-3-amninobenzene, 2-chloro-6-methyl-1-hydroxy-3-aminobenzene, 1,3-diaminobenzene, 6-methoxy-1,3-diaminobenzene, 6-hydroxyethoxy- 1,3 -diaminobenzene, 6-methoxy-5-ethyl- 1,3 -diaminobenzene, 6-ethoxy-1,3-diaminobenzene, 1-bis(beta-hydroxyethyl)amino-3-aminobenzene, 2-methyl- 1,3-diaminobenzene, 6-methoxy- 1-amino-3-[(beta-hydroxyethyl)amino]-benzene, 6-(beta-aminoethoxy)-1,3-diaminobenzene, 6-(beta-hydroxyethoxy)-1-amino-3-(methylamino)benzene, 6-carboxymethoxy-1,3-diaminobenzene, 6-ethoxy-1-bis(beta-hydroxyethyl)amino-3-aminobenzene, 6-hydroxyethyl- 1,3-diaminobenzene, 1-hydroxy-2-isopropyl-5-methylbenzene, 1,3 -dihydroxybenzene, 2-chloro- 1,3-dihydroxybenzene, 2-methyl-1,3-dihydroxybenzene, 4-chloro- 1,3-dihydroxybenzene, 5,6-dichloro-2-methyl- 1,3-dihydroxybenzene, 1-hydroxy-3-amino-benzene, 1-hydroxy-3-(carbamoylmethylamino)benzene, 6-hydroxybenzomorpholine, 4-methyl-2,6-dihydroxypyridine, 2,6-dihydroxypyridine, 2,6-diaminopyridine, 6-aminobenzomorpholine, 1-phenyl-3-methyl-5-pyrazolone, 1-hydroxynaphthalene, 1,7-dihydroxynaphthalene, 1,5-dihydroxynaphthalene, 5-amino-2-methyl phenol, 4-hydroxyindole, 4-hydroxyindoline, 6-hydroxyindole, 6-hydroxyindoline, and mixtures thereof.

Preferred couplers include resorcinol, 1-naphthol, 5-amino-o-cresol, 2-methylresorcinol, m-aminophenol, m-phenylenediamine, 1-phenyl-3-methyl-pyrazol-5-one, their salts, or mixtures The surfactants may be anionic, cationic, nonionic, or amphoteric, as mentioned above.

### SECONDARY THICKENING AGENT

Preferably, the hair dye mixture contains a secondary thickening agent as defined above with respect to the developer and in the same amounts.

### OTHER INGREDIENTS

The hair dye mixture may contain other ingredients such as conditioners, antioxidants, chelating agents, humectants, buffers, conditioining agents, and the like. Preferably the hair dye mixture is free of nonoxynol, and anionic alkyl sulfate and alkyl ether sulfate surfactants, as well as amphoteric and zwitterionic surfactants. The hair dye mixture will contain small amounts of soap, i.e. formed by the reaction of oleic acid and monoethanolamine, to form MEA oleate.

Preferred conditioning agents for use in the hair dye mixture are the nonionic conditioners mentioned above with respect to the developer. In addition, additional conditioning agents such as protein derivatives are preferred. Generally about 0.001-10%, preferably 0.005-8%, more preferably 0.01-5% by weight of the total composition of protein derivatives is suggested. Examples of protein derivatives are hydrolyzed wheat protein, hydrolyzed wheat protein/wheat oligosaccharides, hydrolyzed marine collagen, wheat amino acids, PVP/hydrolyzed wheat protein copolymer (which is a copolymer of polyvinylpyrrolidone and hydrolyzed wheat protein). Particularly preferred is a copolymer of PVP and hydrolyzed wheat protein sold by Croda under the tradename Hydrotriticum PVP.

It may also be desireable to include various plant extracts in the hair dye mixture for extra conditioning and humectancy. Suggested ranges are 0.001-10%, preferably 0.005-8%, more preferably 0.01-5% by weight of the total composition. A preferred plant extract is a mixture of hypnea musciform extract/elidiela acerosa extract/sargassum filipendula extract/sorbitol which is known by the CTFA name algae extract and sorbitol, which is distributed by International Sourcing Inc., in Upper Saddle River, N.J.

Two Component Kit

The invention further comprises a two component kit for the oxidative dyeing of hair. The kit comprises two containers. The first container is filled with:

(a) an aqueous lotion having a pH of 7 to 11, comprising, by weight of the total composition:

(i) about 0.0001-20% (combined weight) of at least one primary intermediate and at least one coupler for the formation of oxidation dyes,

(ii) about 0.5-20% of a nonionic surfactant, and

(iii) about 30-90% water.

The second container is filled with:

(b) a developer composition having a pH of 2.5 to 6.9, comprising, by weight of the total composition:

(i) about 0.5-45% hydrogen peroxide,

(ii) 0.01-20% of a primary thickening agent which is an aqueous solution of acrylic polymer comprised of monomers A and B wherein the A monomer is selected from the group consisting of acrylic acid, methacrylic acid, and mixtures thereof, and the B monomer is selected from the group consisting of a C₁₋₂₂ alkyl methacrylate, and a C₁₋₂₂ alkyl acrylate and mixtures thereof;

(iii) 0.01-20% of a nonionioc surfactant.

(iv) 65-99.9% water.

The lotion and developer may contain other ingredients, as mentioned above. Generally, both the developer and the lotion have a viscosity ranging from about 10 to 200, preferably 10 to 100 centipoise at 25°C. Immediately prior to use, the lotion having a basic pH and the developer, having an acid pH are mixed in approximately about a 1 to 1 ratio and applied to the hair for the appropriate period of time, generally 5 to 60 minutes. The lotion neutralizes the developer thereby causing the primary thickening agent to “swallow” water, and thereby gel the mixture. The mixture is then rinsed from the hair with water.

Method For Dyeing Hair The invention also comprises a method for oxidative dyeing of hair comprising the steps of:

(a) applying to the hair a composition obtained by mixing Composition A and Composition B, wherein Composition A is an aqueous lotion having a pH of 7 to 11, comprising, by weight of the total composition:

(i) about 0.0001-20% (combined weight) of at least one primary intermediate and at least one coupler for the formation of oxidation dyes,

(ii) about 0.5-20% of a nonnionic surfactant, and

(iii) about 10-65% water.

and Composition B is a developer composition having a pH of 2.5 to 6.9 comprising, by weight of the total composition:

(i) about 0.5-45% hydrogen peroxide,

(ii) 0.01-20% of a primary thickening agent comprised of an aqueous solution of acrylic copolymer comprised of monomers A and B wherein the A monomer is selected from the group consisting of acrylic acid, methacrylic acid, and mixtures thereof; and the B monomer is selected from the group consisting of a C₁₋₂₂ alkyl methacrylate, and a C₁₋₂₂ alkyl acrylate and mixtures thereof;

(iii) 0.01-20% of a nonionic surfactant, and

(iv) 65-99.9% water.

b) leaving said composition on the hair for 2 to 60 minutes,

c) rinsing the hair with water.

Preferably the compositions and methods of the invention are free of nonoxynol and alkyl sulfate, alkyl ether sulfate, amphoteric, and zwitterionic surfactants.

The invention will be further described in connection with the following examples which are set forth for the purposes of illustration only.

### EXAMPLE 1

A developer composition for use in oxidative coloration of hair was made according to the following formula:

| | w/w % | | |
|---|---|---|---|
| | 1 | 2 | |
| Water | 77.15 | 78.95 | |
| EDTA | 0.02 | 0.02 | (chelating agent) |
| Methyl paraben | 0.05 | — | (preservative) |
| Steareth-10 allyl ether/acrylates | 0.60 | — | (thickening agent) |
| copolymer | | | |
| Hydrogen peroxide | 9.00 | 9.00 | |
| Trimethylsilylamodimethicone/ | 0.10 | — | (conditioning agent) |
| C11-15 pareth-7/laureth-9/ | | | |
| trideceth-12 glycerin/water | | | |
| Acrylates copolymer | 3.60 | 4.00 | (thickening agent) |
| Lauryl polyglucoside | 9.00 | 8.0 | (surfactant) |
| Phosphoric acid | 0.03 | 0.03 | (pH adjuster) |

The composition was made by combining the ingredients and mixing well.

### EXAMPLE 2

A hair color composition in accordance with the invention was made according to the following formula:

| | | w/w % | |
|---|---|---|---|
| 1 | Water | 76.45 | |
| 2 | Erythrobic acid | 0.20 | (antioxidant) |
| 3 | Sodium sulfite | 0.50 | (antioxidant) |
| 4 | Ethoxydiglycol | 0.50 | (solvent for dye molecules) |
| 5 | Propylene glycol | 2.50 | (humectant) |
| 6 | Tetra sodium EDTA (38% | 0.40 | (chelating agent) |
| | active) | | |
| 7 | Dye | 1.65 | |
| 8 | Oleic acid | 2.00 | (buffer) |
| 9 | Monoethanolamine | 6.40 | (alkalizing agent) |
| 10 | Lauryl polyglycoside | 2.00 | (nonionic surfactant) |
| 11 | Hypnea musciform extract/ | 0.50 | (humectant) |
| | elidiela acerosa extract | | |
| | sargassum filipendula extract/ | | |
| | sorbitol | | |
| 12 | Hydrolyzed marine collagen | 2.50 | (conditioner) |
| 13 | Wheat amino acids | 0.50 | (conditioner) |
| 14 | Hydrolyzed wheat protein/wheat | 0.60 | (conditioner) |
| | oligosaccharides | | |
| 15 | Dimethicone copolyol/ | 0.40 | (conditioner) |
| | dimethicone cyclomethicone | | |
| 16 | PVP/hydrolyzed wheat protein | 0.30 | (conditioner) |
| | copolymer | | |
| 17 | Fragrance | 1.00 | |
| 18 | Polysorbate-20 | 1.00 | (nonionic surfactant) |

Ingredients 1-7 were mixed with heat under a nitrogen blanket. Ingredients 8 and 9 were added. The mixture was cooled and ingredients 10-16 were added. Separately, ingredients 17 and 18 were pre-mixed and added to the composition.

### EXAMPLE 3

Hair was colored using the compositions set forth in Examples 1 and 2. The compositions were mixed in a 1:1 ratio and applied to the hair for 35 minutes. The mixture was rinsed from the hair with water until the hair was completely clean.

### EXAMPLE 4

Hair dye compositions were made according to the following formulas:

| | | w/w % | | | |
|---|---|---|---|---|---|
| | | 1 | 2 | 3 | 4 |
| 1 | Water | 70.2 | 77.8 | 76.8 | 78.4 |
| 2 | Tetrasodium EDTA (38% active) | 0.4 | 0.4 | 0.4 | 0.4 |
| 3 | Sodium sulfite | 0.5 | 0.5 | 0.5 | 0.5 |
| 4 | Erythorbic acid | 0.2 | 0.2 | 0.2 | 0.2 |
| 5 | Propylene glycol | 1.0 | 3.0 | 2.5 | 2.0 |
| 6 | Hexylene glycol | 1.0 | 0.5 | — | 0.5 |
| 7 | Ethoxydiglycol | 1.0 | 0.4 | 0.4 | 0.8 |
| 8 | Dye | 0.07 | 0.8 | 1.5 | 0.7 |
| 9 | Oleic acid | — | 2.0 | 0.3 | — |
| 10 | Monoethanolamine | 3.2 | 6.2 | 6.2 | — |
| 11 | Aminomethylpropanol | 3.0 | — | — | 6.2 |
| 12 | Lactic acid | 2.0 | — | — | 2.0 |
| 13 | Lauryl polyglucoside | 2.0 | 2.0 | 2.0 | 2.0 |
| 14 | Seaweed algae extract/sorbitol | 0.6 | 0.5 | 0.5 | 0.5 |
| 15 | Hydrolyzed marine collagen | 2.0 | 2.0 | 2.5 | 2.0 |
| 16 | Wheat amino acids | 0.5 | 0.5 | 0.5 | 0.5 |
| 17 | Hydrolyzed wheat protein/wheat | 0.5 | 0.4 | 0.5 | 0.6 |
| | oligosaccharides | | | | |
| 18 | Dimethicone copolyol/dimethicone/ | 0.4 | 0.3 | 0.4 | 0.4 |
| | cyclomethicone | | | | |
| 19 | PVP/hydrolyzed wheat protein | 0.4 | 0.3 | 0.4 | 0.2 |
| | copolymer | | | | |
| 20 | Fragrance | 1.0 | 1.0 | 1.0 | 1.0 |
| 21 | Polysorbate 20 | 1.0 | 1.0 | 1.0 | 1.0 |

Ingredients 1-7 were heated to 85°C. and mixed well. When all solids were dissolved, 8 was added, followed by 9. The composition was mixed until clear. The heat was removed and while the mixture cooled to 40°C. ingredients 10-19 were added. Ingredients 20 and 21 were separately mixed and added at end.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. A developer composition having a pH of 2.5 to 6.9, for use in oxidative dyeing of hair comprising, by weight of the total composition:
0.01-20% of a primary thickening agent which is an aqueous solution of acrylic polymer comprised of A and B monomers wherein the A monomer is selected from the group consisting of acrylic acid, methacrylic acid, and mixtures thereof; and the B monomer is selected from the group consisting of a C₁₋₂₂ alkyl methacrylate, and a C₁₋₂₂ alkyl acrylate, and mixtures thereof;
0.01-20% of a nonionic surfactant,
0.01-25% hydrogen peroxide, and
65-99.9% water

2. The composition of claim 1 where in the primary thickening agent the B monomer is a C₁₋₄ alkyl acrylate, a C₁₋₄ alkyl methacrylate, or mixtures thereof.

3. The composition of claim 1 wherein the acrylic polymer solution has a solids content of 25 to 45 percent by weight of the total solution, a pH of 2 to 4, and a density of 1.04 to 1.08.

4. The composition of claim 1 wherein the nonionic surfactant comprises an alkylglycoside having the general formula:
R₁—O—(R₂O)ₜ—(G)ₙ—H
wherein R₁ is a linear or branched alkyl or alkenyl radical having 12 to 30 carbon atoms, R₂ is a C₂₋₄ alkylene, (G) is an anhydroglucose unit, t is a number between 0 and 10, preferably 0 to 4, and n is a number from about 1 to 15.

5. The composition of claim 4 wherein R₁ is C₁₆, t is 0, and n ranges from 1 to 5, and the compound is lauryl polyglucose.

6. The composition of claim 1 further comprising 0.01-10% by weight of the total composition of a secondary thickening agent comprised of polymer of A, B, and C monomers wherein the A monomer is selected from the group consisting of acrylic acid, methacrylic acid, and mixtures thereof; and the B monomer is selected from the group consisting of a C₁₋₄ alkyl methacrylate, and a C₁₋₄ alkyl acrylate, and mixtures thereof; and C has the general formula: 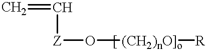
wherein Z is —(CH₂)ₘ— or 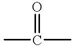
wherein m is 1-10, n is 2-3, o is 2-200, and R is a C₁₀₋₃₀ straight or branched chain alkyl.

7. The composition of claim 6 where in the C monomer Z is (CH₂)ₘ.

8. The composition of claim 7 wherein the secondary thickening agent is steareth-10 allyl ether/acrylates copolymer.

10. The composition of claim 1 addition comprising 0.001-10% by weight of the total composition of a nonionic silicone conditioning agent.

11. The composition of claim 10 wherein the nonionic silicone conditioning agent is trimethylsilylamodimethicone.

12. A mixture for oxidation dyeing of hair comprising, by weight of the total composition:
(a) about 0.0001-20% (combined weight) of at least one primary intermediate and at least one coupler for the formation of oxidation dyes,
(b) about 0.5-30% of a nonionic surfactant,
(c) about 0.01-20% of a primary thickening agent which is an acrylic copolymer comprised of monomers A and B wherein the A monomer is selected from the group consisting of acrylic acid, methacrylic acid, and mixtures thereof; and the B monomer is selected from the group consisting of a C₁₋₂₂ alkyl methacrylate, and a C₁₋₂₂ alkyl acrylate and mixtures thereof, and
(d) about 40-99% water.

13. The composition of claim 12 wherein the nonionic surfactant comprises an alkylglycoside having the general formula:
R₁—O—(R₂O)ₜ—(G)ₙ—H
wherein R₁ is a linear or branched alkyl or alkenyl radical having 12 to 30 carbon atoms, R₂ is a C₂₋₄ alkylene, (G) is an anhydroglucose unit, t is a number between 0 and 10, preferably 0 to 4, and n is a number from about 1 to 15.

14. The composition of claim 12 wherein the mixture further comprises 0.01-10% by weight of the total composition of a secondary thickening agent comprised of polymer of A, B, and C monomers wherein the A monomer is selected from the group consisting of acrylic acid, methacrylic acid, and mixtures thereof; and the B monomer is selected from the group consisting of a C₁₋₂₂ alkyl methacrylate, and a C₁₋₂₂ alkyl acrylate, and mixtures thereof; and C has the general formula: 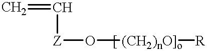
wherein Z is —(CH₂)ₘ— or 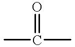
wherein m is 1-10, n is 2-3, o is 2-200, and R is a C₁₀₋₃₀ straight or branched chain alkyl.

15. The composition of claim 12 further comprising 0.001-10% by weight of the total composition of a protein derivatives selected from the group consisting of hydrolyzed wheat protein, hydrolyzed wheat protein/wheat oligosaccharides, hydrolyzed marine collagen, wheat amino acids, PVP/hydrolyzed wheat protein copolymer, and mixtures thereof.

16. The composition of claim 12 further comprising 0.001-10% of a silicone conditioning agent.

15. The composition of claim 16 wherein the silicone conditioning agent is trimethylsilylamodimethicone.

16. A two component kit for oxidative dyeing of hair comprising:
(a) an aqueous lotion having a pH of 7 to 11, comprising, by weight of the total composition:
(i) about 0.0001-20% (combined weight) of at least one primary intermediate and at least one coupler for the formation of oxidation dyes,
(ii) about 0.5-20% of a nonnionic surfactant, and
(iii) about 30-90% water.
(b) a developer composition having a pH of 2.5 to 6.9, comprising, by weight of the total composition:
(i) about 0.5-45% hydrogen peroxide,
(ii) 0.01-20% of a primary thickening agent which is an aqueous solution of acrylic polymer comprised of monomers A and B wherein the A monomer is selected from the group consisting of acrylic acid, methacrylic acid, and mixtures thereof; and the B monomer is selected from the group consisting of a C₁₋₂₂ alkyl methacrylate, and a C₁₋₂₂ alkyl acrylate and mixtures thereof;
(iii) 0.01-20% of a nonionic surfactant,
(iv) 65-99.9% water.

17. The kit of claim 16 wherein the developer further comprises comprises 0.01-10% by weight of the total composition of a secondary thickening agent comprised of polymer of A, B, and C monomers wherein the A monomer is selected from the group consisting of acrylic acid, methacrylic acid, and mixtures thereof; and the B monomer is selected from the group consisting of a C₁₋₂₂ alkyl methacrylate, and a C₁₋₂₂ alkyl acrylate, and mixtures thereof, and C has the general formula: 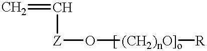
wherein Z is —(CH₂)ₘ or 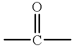
wherein m is 1-10, n is 2-3, o is 2-200, and R is a C₁₀₋₃₀ straight or branched chain alkyl.

18. The kit of claim 17 wherein the nonionic surfactant in the developer and the lotion is an alkylglycoside having the general formula:
R₁—O—(R₂O)ₜ—(G)ₙ—H
wherein R₁ is a linear or branched alkyl or alkenyl radical having 12 to 30 carbon atoms, R₂ is a C₂₋₄ alkylene, (G) is an anhydroglucose unit, t is a number between 0 and 10, preferably 0 to 4, and n is a number from about 1 to 15.

19. A method for oxidative dyeing of hair comprising the steps of:
(a) applying to the hair a composition obtained by mixing Composition A and Composition B, wherein Composition A is an aqueous lotion comprising, by weight of the total composition:
(i) about 0.0001-20% (combined weight) of at least one primary intermediate and at least one coupler for the formation of oxidation dyes,
(ii) about 0.5-20% of a nonnionic surfactant, and
(iii) about 10-65% water.
and Composition B is a developer composition comprising, by weight of the total composition:
(i) about 0.5-45% hydrogen peroxide,
(ii) 0.01-20% of a primary thickening agent comprised of an aqueous solution of acrylic copolymer comprised of monomers A and B wherein the A monomer is selected from the group consisting of acrylic acid, methacrylic acid, and mixtures thereof; and the B monomer is selected from the group consisting of a C₁₋₂₂ alkyl methacrylate, and a C₁₋₂₂ alkyl acrylate and mixtures thereof;
(iii) 0.01-20% of a nonionic surfactant,
(iv) 65-99.9% water.
b) leaving said composition on the hair for 2 to 60 minutes,
c) rinsing the hair with water.

20. The method of claim 19 wherein the developer and lotion compositions are free of nonoxynol, alkyl sulfate surfactants, alkyl ether sulfate surfactants, zwitterionic surfactants, and amphoteric surfactants.
